(19) United States
(12) Patent Application Publication
(10) Pub. No.: US 2007/0259857 A1
(43) Pub. Date: Nov. 8, 2007

(54) Amorphous form of Olanzapine

(76) Inventor: Jason Gray, Hertfordshire (GB)

Correspondence Address: WOLF GREENFIELD & SACKS, P.C., 600 ATLANTIC AVENUE, BOSTON, MA, 02210-2206, US

(21) Appl. No.: 10/561,009

(22) PCT Filed: Jun. 15, 2004
(86) PCT No.: PCT/GB04/02579

§ 371 Date: Jun. 21, 2007

(30) Foreign Application Priority Data
Jun. 18, 2003 (GB) 0314149.6

(51) Int. Cl. A61K 31/551; A61P 1/00; A61P 25/00; A61P 25/18; A61P 25/24; C07D 243/10

(52) U.S. Cl. 514/220; 540/557

(57) ABSTRACT

The present invention relates to an amorphous form of olanzapine Formula (I): and a process for its preparation. The present invention further relates to a pharmaceutical composition comprising an amorphous form of olanzapine (I). The pharmaceutical composition may be used, in particular, for the treatment of psychiatric, psychological or psychotic disorders, anxiety disorders, or gastrointestinal or functional bowel disorders. The present invention also relates to a method of treating said disorders.

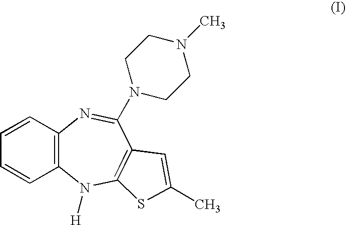

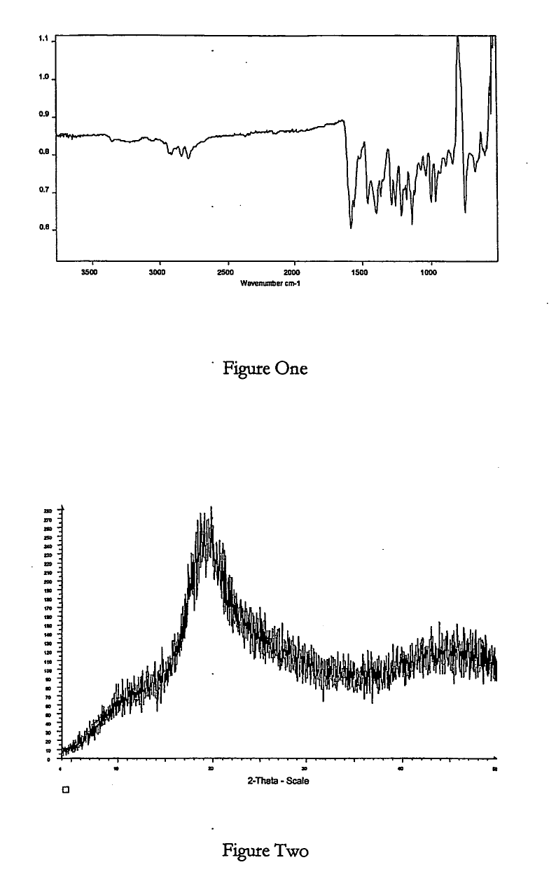

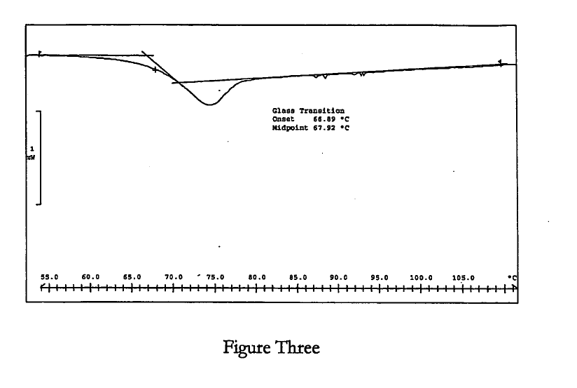

### BACKGROUND ART

The present invention relates to an amorphous form of olanzapine and a process for its preparation. The present invention further relates to a pharmaceutical composition comprising an amorphous form of olanzapine. The pharmaceutical composition may be used, in particular, for the treatment of psychiatric, psychological or psychotic disorders, anxiety disorders, or gastrointestinal or functional bowel disorders. The present invention also relates to a method of treating said disorders.

### TECHNICAL FIELD

The present invention relates to a novel amorphous form of the antipsychotic drug olanzapine, 2-methyl-4-(4-methyl-1-piperazinyl)-10H-thieno[2,3-b][1,5]benzodiazepine (I), processes for preparing such a form, compositions comprising such a form, and uses for such a form and compositions.

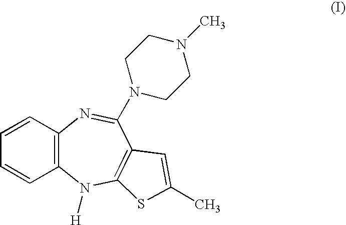

The manufacturing process for many pharmaceuticals is hindered by the fact that the organic compound, which is the active drug substance, has an irregular crystalline form. In some cases, such irregularities can cause handling difficulties during the manufacturing process and/or undesirable properties being imparted to the final drug or dosage form. The latter include inconsistent bioavailability, solubility and dissolution rates.

Olanzapine was originally described as a metastable crystalline product, referred to in patent U.S. Pat. No. 5,736,541 as Form I, wherein a more stable crystalline form, referred to as Form II was also disclosed. Crystalline alcohol solvates of olanzapine have also been disclosed in patent U.S. Pat. No. 5,703,232, and further crystalline forms of olanzapine, referred to as Forms III, IV and V respectively, have been disclosed in patent U.S. Pat. No. 6,348,458.

Difficulties may ensue if the pharmaceutical material contains mixtures of polymorphs, especially if the different polymorphs have varying physical properties. An amorphous form of a drug may have the particular advantages of inter alia (a) having improved bio-efficacy as a result of the higher solubility and dissolution rate etc., and/or (b) being overall more constant than in polymorphic form.

It has been surprisingly found that olanzapine has an amorphous form for which the glass transition temperature (Tg) is ˜66° C. The fact that this temperature is so high suggests that when the new material is stored way below this temperature (for example 25° C.), the kinetics of converting to the stable crystalline form would be slow and an amorphous phase would be stable during the shelf life of the product. Consequently the amorphous form of the present invention will be suitable to use as a pharmaceutical and have the advantages over the crystalline forms described earlier.

### SUMMARY OF THE INVENTION

It is an object of the present invention to provide olanzapine in a solid amorphous form that affords the compound improved handling properties and/or improved properties as a pharmaceutical agent.

Therefore, a first aspect of the present invention provides an amorphous form of olanzapine.

The amorphous form in accordance with the invention can be used to advantage in the preparation of pharmaceutical dosage or drug forms. When in particulate form, the amorphous form in accordance with the invention is free flowing and does not present any of the handling difficulties associated with irregularly shaped crystals. It, therefore, can be employed in the manufacture of pharmaceuticals that do not suffer from the problems, such as inconsistent bioavailability, solubility and dissolution rates, that can be manifest in dosage forms manufactured using previously available forms of olanzapine that have irregularly shaped and/or metastable crystals.

The present invention therefore provides an amorphous form of olanzapine. Preferably the amorphous form of olanzapine is in particulate form. Preferably the amorphous form of olanzapine is substantially pure.

In the context of the present application, the term “substantially pure” means that the amorphous form of olanzapine comprises less than 10% of crystalline forms of olanzapine and less than 2% of other impurities. Preferably the amorphous form of olanzapine comprises less than 5% of crystalline forms of olanzapine, more preferably less than 2%, and even more preferably less than 1%. More preferably the amorphous form of olanzapine is substantially free of crystalline forms of olanzapine. Preferably the amorphous form of olanzapine comprises less than 1% of other impurities, more preferably less than 0.5%.

Preferably the amorphous form of olanzapine has an IR spectrum substantially as shown in Figure One, an XRPD spectrum substantially as shown in Figure Two, and/or a DSC trace substantially as shown in Figure Three.

Preferably the amorphous form of olanzapine is suitable for use as a medicament

A second aspect of the present invention provides a process for the preparation of an amorphous form of olanzapine, comprising the step of melting one or more crystalline forms of olanzapine. Preferably the process further comprises the step of cooling the melt.

Alternatively, the present invention provides a process for the preparation of an amorphous form of olanzapine, comprising the step of freeze-drying or spray-drying a solution comprising olanzapine. Preferably the solution comprises water or an alcohol (such as ethanol) or a mixture thereof as the solvent. Alternatively the freeze-drying or spray-drying process is performed in the presence of an excipient such as lactose, povidone or crospovidone. In some cases, forming a solid solution comprising amorphous olanzapine and an excipient is desirable and such solid solutions can be prepared by this embodiment of the process of the invention.

The processes for the preparation of an amorphous form of olanzapine, comprising the step of freeze-drying or spray-drying or forming a solid solution, are particularly suitable for the preparation of amorphous olanzapine on a commercial scale.

In further aspects, the present invention provides a method of preparing a pharmaceutical dosage form that utilises an amorphous form in accordance with the first aspect of the invention. Preferably the pharmaceutical composition prepared by this method is for oral or parenteral administration. Preferably the pharmaceutical composition is a tablet or capsule for oral administration, or a solution for oral or parenteral administration.

The present invention also provides a pharmaceutical dosage form prepared by such a method. The dosage form is preferably solid and comprises, in addition to the amorphous olanzapine, one or more conventional pharmaceutically acceptable excipient(s). Preferred dosage forms in accordance with the invention include tablets, capsules and the like. Tablets can be prepared by conventional techniques, including direct compression, wet granulation and dry granulation. Capsules are generally formed from a hard gelatine material and can include a conventionally prepared granulate of excipients and adduct or solvate in accordance with the invention.

The amorphous form in accordance with the invention may also be useful as precursor to other novel polymorphic forms of olanzapine that may be useful in the preparation of pharmaceutical products.

The present invention also provides the use of an amorphous form of olanzapine in accordance with the first aspect of the invention for the preparation of a medicament, preferably for use in treating psychiatric illnesses, such as psychoses.

The present invention provides a pharmaceutical composition, comprising an amorphous form of olanzapine. The pharmaceutical composition of the present invention may be for immediate, sustained or delayed release. The amorphous form of olanzapine is particularly suitable for an oral disintegrating formulation. Preferably the pharmaceutical composition further comprises a pharmaceutically acceptable carrier, excipient or diluent.

The pharmaceutical composition can be administered by oral, parental (including intravenous, subcutaneous, intramuscular, intradermal, intratracheal, intraperitoneal intraarticular, intracranial and epidural), transdermal, airway (aerosol), rectal, vaginal or topical (including buccal, mucosal and sublingual) administration. Preferably the pharmaceutical composition is for oral or parenteral (in particular intramuscular) administration.

For oral administration, the pharmaceutical composition will generally be provided in the form of tablets, capsules, hard or soft gelatine capsules, caplets, troches or lozenges, as a powder or granules, or as an aqueous solution, suspension or dispersion.

Tablets for oral use may include the active ingredient mixed with pharmaceutically acceptable excipients such as inert diluents, disintegrating agents, binding agents, lubricating agents, sweetening agents, flavouring agents, colouring agents and preservatives. Suitable excipients for tablets are lactose, hydroxypropylcellulose, crospovidone, microcrystalline cellulose, magnesium stearate, gelatine, mannitol, aspartame, sodium methyl p-hydroxybenzoate and sodium propyl p-hydroxybenzoate. If desired, the tablets may be coated with materials such as hypromellose and/or carnauba wax.

Capsules for oral use include hard gelatine capsules in which the active ingredient is mixed with a solid diluent, and soft gelatine capsules wherein the active ingredient is mixed with water or an oil such as peanut oil, liquid paraffin, olive oil or mineral oil.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

For parenteral use, the active ingredient will generally be formulated as a sterile aqueous solution or suspension, buffered to an appropriate pH and isotonicity. Suitable aqueous vehicles include Ringer's solution and isotonic sodium chloride or glucose. Aqueous solutions may comprise lactose, tartaric acid, hydrochloric acid and/or sodium hydroxide. Aqueous suspensions may include suspending agents such as cellulose derivatives, sodium alginate, polyvinylpyrrolidone and gum tragacanth, and a wetting agent such as lecithin. Suitable preservatives for aqueous suspensions include methyl, ethyl and n-propyl p-hydroxybenzoate.

For topical and transdermal administration, the active ingredient will generally be provided in the form of ointments, cataplasms (poultices), pastes, powders, dressings, creams, plasters or patches.

Suitable suspensions and solutions can be used in inhalers for airway (aerosol) administration.

Preferably the pharmaceutical composition is in the form of a tablet or capsule for oral administration or in the form of a powder suitable for preparing a solution for oral or parenteral administration.

Preferably the pharmaceutical composition is in unit dosage form comprising from 0.1 mg to 200 mg amorphous olanzapine, preferably from 0.25 mg to 100 mg, more preferably from 0.5 mg to 50 mg, more preferably from 1 mg to 30 mg, and even more preferably from 2 mg to 20 mg.

The amorphous olanzapine of the present invention is effective over a wide dosage range, the actual dose administered being dependent on the condition being treated. For example, in the treatment of adult humans, dosages from 0.5 mg to 50 mg, preferably from 1 mg to 30 mg, more preferably from 2 mg to 20 mg per day may be used. The desired dose is normally presented once a day, but may be dosed as two, three, four or more sub-doses administered at appropriate intervals throughout the day.

Preferably the pharmaceutical composition is for the treatment of a psychiatric, psychological or psychotic disorder, an anxiety disorder, or a gastrointestinal or functional bowel disorder.

The psychiatric, psychological or psychotic disorder may be schizophrenia, schizophreniform disorder, schizoaffective disorder, Tourette's disorder, mania, manic episode, severe manic episode, delusional disorder, psychotic disorder, psychosis, bipolar disorder, depression, or a disorder of the central nervous system. Preferably the psychiatric, psychological or psychotic disorder is schizophrenia, manic episode, severe manic episode, psychosis, or bipolar disorder.

The anxiety disorder may be generalised anxiety disorder, obsessive-compulsive disorder, post-traumatic stress disorder, or an anxiety state.

The gastrointestinal or functional bowel disorder may be irritable bowel syndrome, gastric hypermotility, ichlasia, hypertonic lower esophageal sphincter, tachygastria, constipation, diarrhoea, mucorrhoea, or pain or discomfort over the course of the sigmoid colon.

A further aspect of the present invention provides a method of treating a condition selected from a psychiatric, psychological or psychotic disorder, an anxiety disorder, or a gastrointestinal or functional bowel disorder, comprising administering an effective amount of an amorphous form of olanzapine, an effective amount of a pharmaceutical composition prepared by a method of the present invention, or an effective amount of a pharmaceutical composition of the present invention, to a patient in need thereof. Preferably the patient is a human.

Preferably the amount of amorphous olanzapine administered is from 0.1 mg to 200 mg per day, preferably from 0.25 mg to 100 mg, more preferably from 0.5 mg to 50 mg, more preferably from 1 mg to 30 mg, and even more preferably from 2 mg to 20 mg.

The psychiatric, psychological or psychotic disorder may be schizophrenia, schizophreniform disorder, schizoaffective disorder, Tourette's disorder, mania, manic episode, severe manic episode, delusional disorder, psychotic disorder, psychosis, bipolar disorder, depression, or a disorder of the central nervous system. Preferably the psychiatric, psychological or psychotic disorder is schizophrenia, manic episode, severe manic episode, psychosis, or bipolar disorder.

The anxiety disorder may be generalised anxiety disorder, obsessive-compulsive disorder, post-traumatic stress disorder, or an anxiety state.

The gastrointestinal or functional bowel disorder may be irritable bowel syndrome, gastric hypermotility, ichlasia, hypertonic lower esophageal sphinctor, tachygastria, constipation, diarrhoea, mucorrhoea, or pain or discomfort over the course of the sigmoid colon.

The present invention is now illustrated, but in no way limited, by the following example and figures.

### BRIEF DESCRIPTION OF THE FIGURES

Figure One is an IR spectrum of amorphous olanzapine. Infra Red (IR) analysis was performed on a Bruker Equinox 55 using a specac diamond ATR system between 4000 cm⁻¹ and 550 cm⁻¹.

Figure Two is an XRPD spectrum of amorphous olanzapine. X-ray powder diffraction (XRPD) was performed on a Bruker D8 advance difractometer, at 25° C. between the angles of 4° and 50° 2 theta.

Figure Three is a DSC trace of amorphous olanzapine. Differential Scanning Calorimetry (DSC) was performed on a Mettler Toledo 821e.

### DETAILED DESCRIPTION OF THE INVENTION

### Example One

Crystalline olanzapine (6.80 g) was weighed into a beaker and placed into an oven at 203° C. to melt After 45 minutes the molten olanzapine was poured into a dish to form a brittle block. The sample was then allowed to cool at room temperature for 3 hours. The sample was ground in a clay mortar and pestle, and analysed by IR (Figure One), XRPD (Figure Two), DSC (Figure Three) and Karl Fisher analysis.

When exposing X-rays to a sample that is in an ordered crystalline lattice, then a series of diffraction lines is obtained giving a characteristic “finger print” for that lattice. This is the main technique to compare different crystalline polymorphic forms of a compound. However, if the sample is completely in an amorphous state (i.e. not in a regular lattice), then no such diffraction lines will be observed, and a very diffuse broad band will be observed relating to scattering of X-rays from the sample.

Figure Two shows the XRPD pattern for the sample prepared in Example One and no diffraction is observed, with just a broad diffuse band indicating the sample is in an amorphous form.

DSC analysis of the sample exhibited a glass transition at approximately 66° C., indicating that the sample was amorphous (see Figure Three).

Karl Fischer analysis indicated that the sample contained no water and was not a hydrated form. Karl Fischer analysis was performed using a Mettler Toledo DL53 under standard conditions.

The sample of ground olanzapine was stored at room temperature for 13 weeks for stability testing using XRPD analysis. No difference was observed in spectral XRPD analysis of the original sample and the sample that had been kept in storage for 13 weeks at room temperature.

It can be concluded that by the melting of crystalline olanzapine and subsequent cooling an amorphous phase can be formed. Due to the surprisingly relatively high Tg of ˜66° C., nucleation and crystallisation is slow and not observed even after coarse grinding and prolonged storage.

It will be understood that the present invention has been described above by way of example only. The example is not intended to limit the scope of the invention. Various modifications and embodiments can be made without departing from the scope and spirit of the invention, which is defined by the following claims only.

1. An amorphous form of olanzapine.

2. An amorphous form of olanzapine as claimed in claim 1, wherein the olanzapine is in particulate form.

3. An amorphous form of olanzapine as claimed in claim 1, wherein the olanzapine is substantially pure.

4. An amorphous form of olanzapine as claimed in claim 1, having an IR spectrum substantially as shown in Figure One, an XRPD spectrum substantially as shown in Figure Two, and/or a DSC trace substantially as shown in Figure Three.

5. An amorphous form of olanzapine as claimed in claim 1, for use as a medicament.

6. A process for the preparation of an amorphous form of olanzapine as claimed in claim 1, comprising the step of melting one or more crystalline forms of olanzapine.

7. A process as claimed in claim 6, further comprising the step of cooling the melt.

8. A process for the preparation of an amorphous form of olanzapine as claimed in claim 1, comprising the step of freeze-drying or spray drying a solution comprising olanzapine.

9. A process as claimed in claim 8, wherein the solution further comprises water or an alcohol or a mixture thereof.

10. A process for the preparation of an amorphous form of olanzapine as claimed in claim 1, comprising the step of forming a solid solution comprising olanzapine and an excipient.

11. A process as claimed in claim 10, wherein the excipient is lactose, povidone or crospovidone.

12. A method of preparing a pharmaceutical composition, comprising utilizing an amorphous form of olanzapine as claimed in claim 1.

13. A method as claimed in claim 12, wherein the pharmaceutical composition is for oral or parenteral administration.

14. A method as claimed in claim 12, wherein the pharmaceutical composition is a tablet or capsule for oral administration.

15. A method as claimed in claim 12, wherein the pharmaceutical composition is a solution for oral or parenteral administration.

16. A pharmaceutical composition, comprising an amorphous form of olanzapine as claimed in claim 1.

17. A pharmaceutical composition as claimed in claim 16, further comprising a pharmaceutically acceptable carrier, excipient or diluent.

18. A pharmaceutical composition as claimed in claim 16, wherein the composition is for oral or parenteral administration.

19. A pharmaceutical composition as claimed in claim 16, wherein the composition is in the form of a tablet or capsule for oral administration or in the form of a powder suitable for preparing a solution for oral or parenteral administration.

20. A pharmaceutical composition as claimed in claim 19, wherein the composition is in the form of a tablet or capsule and further comprises lactose, hydroxypropylcellulose, crospovidone, microcrystalline cellulose, magnesium stearate, gelatin and/or manitol.

21. A pharmaceutical composition as claimed in claim 19, wherein the composition is in the form of a powder and further comprises lactose, tartaric acid, hydrochloric acid and/or sodium hydroxide.

22. A pharmaceutical composition as claimed in claim 16, wherein the composition is in unit dosage form comprising from 1 mg to 30 mg amorphous olanzapine.

23. A pharmaceutical composition as claimed in claim 16, for the treatment of a psychiatric, psychological or psychotic disorder, an anxiety disorder, or a gastrointestinal or functional bowel disorder.

24. A pharmaceutical composition as claimed in claim 23, wherein the psychiatric, psychological or psychotic disorder is schizophrenia, schizophreniform disorder, schizoaffective disorder, Tourette's disorder, mania, manic episode, severe manic episode, delusional disorder, psychotic disorder, psychosis, bipolar disorder, depression, or a disorder of the central nervous system.

25. A pharmaceutical composition as claimed in claim 24, wherein the psychiatric, psychological or psychotic disorder is schizophrenia, manic episode, severe manic episode, psychosis, or bipolar disorder.

26. A pharmaceutical composition as claimed in claim 23, wherein the anxiety disorder is generalized anxiety disorder, obsessive-compulsive disorder, post-traumatic stress disorder, or an anxiety state.

27. A pharmaceutical composition as claimed in claim 23, wherein the gastrointestinal or functional bowel disorder is irritable bowel syndrome, gastric hypermotility, ichlasia, hypertonic lower esophageal sphincter, tachygastria, constipation, diarrhoea, mucorrhoea, or pain or discomfort over the course of the sigmoid colon.

28. A method of treating a condition selected from a psychiatric, psychological or psychotic disorder, an anxiety disorder, or a gastrointestinal or functional bowel disorder, comprising administering an effective amount of an amorphous form of olanzapine as claimed in claim 1 to a patient in need thereof.

29. A method as claimed in claim 28, wherein the patient is a human.

30. A method as claimed in claim 28, wherein the amount of an amorphous form of olanzapine administered is from 2 mg to 20 mg per day.

31. A method as claimed in claim 28, wherein the psychiatric, psychological or psychotic disorder is schizophrenia, schizophreniform disorder, schizoaffective disorder, Tourette's disorder, mania, manic episode, severe manic episode, delusional disorder, psychotic disorder, psychosis, bipolar disorder, depression, or a disorder of the central nervous system.

32. A method of claim 31, wherein the psychiatric, psychological or psychotic disorder is schizophrenia, manic episode, severe manic episode, psychosis, or bipolar disorder.

33. A method as claimed in claim 28, wherein the anxiety disorder is generalized anxiety disorder, obsessive-compulsive disorder, post-traumatic stress disorder, or an anxiety state.

34. A method as claimed in claim 28, wherein the gastrointestinal or functional bowel disorder is irritable bowel syndrome, gastric hypermotility, ischlasia, hypertonic lower esophageal sphincter, tachygastria, constipation, diarrhoea, mucorrhoea, or pain or discomfort over the course of the sigmoid colon.
